(19) United States
(12) Patent Application Publication
(10) Pub. No.: US 2003/0114928 A1
(43) Pub. Date: Jun. 19, 2003

(54) Intraocular lens implant

(76) Inventor: Graham David Barrett, City Beach (AU)

Correspondence Address: FISH & NEAVE, 1251 AVENUE OF THE AMERICAS, 50TH FLOOR, NEW YORK, NY, 10020-1105, US

(21) Appl. No.: 10/298,309

(22) Filed: Nov. 14, 2002

Related U.S. Application Data
(63) Continuation of application No. PCT/AU01/00578, filed on May 18, 2001.

(51) Int. Cl. A61F 2/16

(52) U.S. Cl. 623/6.59

(57) ABSTRACT

A dehydrated intraocular lens implant is first folded and then inserted into the eye through an incision in the eye. The folded dehydrated intraocular lens implant is then allowed to unfold, hydrate in the eye and expand to its desired dimensions. The intraocular lens implant is comprised of a polymer, wherein the polymer is flexible and elastic when dehydrated so as to facilitate the intraocular lens implant to be folded and inserted into the incision in the eye. The polymer also expansile when hydrated, such that after insertion into the eye, the intraocular lens implant hydrates and expands.

### FIELD OF THE INVENTION

The present invention relates to intraocular lens implants.

### BACKGROUND OF THE INVENTION

The crystalline lens is a transparent structure that focuses light in the human eye. Opacification of the lens known as cataract formation is a common cause of poor vision in the elderly and can be corrected surgically. Modern cataract surgery is performed by manual extracapsular cataract extraction or by phacoemulsification. Manual extracapsular cataract extraction involves expressing the hard nucleus of the cataract through a 10 mm to 12 mm incision. Phacoemulsification utilises ultrasonic energy transmitted by a needle to fragment the nucleus and allow aspiration of the cataract through a 2.5 mm to 3.2 mm incision.

In both operations an opening is made in the anterior capsule to allow removal of the lens contents. The capsular bag remnant, however, is left in situ to provide support for an intraocular lens implant which is inserted following removal of the cataract to replace the focusing power of the natural crystalline lens.

An intraocular lens implant typically comprises a centre focusing element known as the optic and a peripheral support structure known as the haptic. The optic and the haptic of the intraocular lens implant may be manufactured from transparent rigid plastics material such as polymethyl methacrylate or from flexible plastics materials such as silicone or hydrogel polymers. Intraocular lens implants manufactured from flexible materials are preferable to those made of rigid materials because the flexible lens may be folded to allow insertion through a small incision in the sclera or outercoat of the eye. The inserted folded lens is then required to unfold to its original configuration.

A small incision is desirable in cataract surgery to avoid distortion of the corneal curvature known as astigmatism. This results in faster visual rehabilitation and better unaided visual acuity following surgery. Studies have demonstrated that an incision size of less than 2.5 mm does not induce significant astigmatism. A smaller incision is also safer during surgery and allows faster wound healing following surgery. A small incision is also less susceptible to traumatic wound disruption in the postoperative period.

Until recently the incision size required to remove a cataract with phacoemulsification was 3.2 mm. This allowed leakage of fluid around the sleeve of the phacoemulsification needle which cooled the needle and avoided thermal injury to the sclera or cornea. However, a new phacoemulsification needle designed by the present inventor allows phacoemulsification through a 2.5 mm incision reducing wound leakage and reducing the chance of thermal injury. Grooves along the shaft of the needle cool the needle and allow increased infusion to the eye. The reduction in leakage of fluid around the sleeve and the improved infusion enhances chamber stability and improves the safety of the phacoemulsification procedure.

The present inventor has also designed an irrigation aspiration cannula for use with incisions smaller than the conventional incision size of 3.2 mm which also facilitates the removal of residual cortical material. Recently, there has been interest in energy sources other than ultrasound to remove cataracts through incisions of less than 2.5 mm. Suggested techniques include laser, impeller and hydrojet technologies and may require a two port procedure to achieve adequate infusion.

It can be seen therefore that advances in surgical technique have reduced the required incision size for phacoemulsification from 3.2 to 2.5 mm. Conventional foldable intraocular lens implants, however, require at least a 2.8 mm incision. The minimum incision size required depends on the structure and material of an intraocular lens implant as well as the method of insertion used to insert the intraocular lens implants.

The size of the incision required to insert an intraocular lens implant is largely determined by the dimensions of the optic. The minimum recommended diameter of an intraocular lens implant optic is 5.5 mm. Optic diameters less than 5.5 mm have been associated with a higher incidence of reflections from the edge of the optic. Intraocular lenses with an optic diameter less than 5.5 mm are also susceptible to unwanted edge glare due to minor decentration of the lens which may occur in the postoperative period with shrinkage of the capsular bag. The center thickness and edge thickness of the intraocular lens implant is related to the optic diameter and also the refractive index of the optic material.

Flexible implants with a high refractive index have been introduced with the aim of reducing incision size. A typical intraocular lens implant with an optic of 5.5 mm has a center thickness ranging from 0.6 to 1.2 mm depending on the dioptric power, refractive index and edge thickness of the intraocular lens implant. The higher the power the thicker the intraocular lens implant and the larger the incision size required to insert the lens implant. An aspheric optic may also reduce the centre thickness for a lens implant of a given refractive index and optic diameter. A conventional flexible lens with a dioptric power of 21.5, a 5.5 mm optic diameter and high refractive index of 1.52 requires an incision size of at least 3.00 mm when folded.

Injector systems which roll and compress the lens can be inserted through smaller incisions. Although it is asserted that injectors are capable of inserting flexible implants through incision sizes as low as 2.5 mm studies have demonstrated that the final incision size is invariably larger than the alleged incision size due to undesireable stretching of the wound. Furthermore, injector systems may result in damage to the implant lens due to extreme compression when injecting the implant lens through the tight incision.

Intraocular implant lenses manufactured from hydrogel lenses have been described which are inserted in the unfolded dehydrated state. The dimensions of the optic of a dehydrated implant lens are less than the fully hydrated dimensions and thus the implant lens can be inserted through a smaller incision than otherwise required. The difference between the dehydrated and hydrated dimensions is known as the swell ratio and is related to the water content of the material. The refractive index is also related to the water content. A hydrogel with a water content of 38% has a swell ratio of 1.2 and refractive index of 1.44. A dehydrated implant lens with a 5.5 mm optic in the fully hydrated state would thus require at least a 5.00 mm incision when dehydrated as the thickness of the implant lens is typically 0.8 mm when dehydrated. A higher water content hydrogel material eg. 70% by weight, has a swell ratio of approximately 2. The refractive index of the material however is considerably lower at 1.41 and the thickness of the dehydrated lens is still in the order of 0.8 mm. The required incision size is still expected to be greater than 3.00 mm. The required incision size of expansile intraocular lenses inserted unfolded is no smaller than that achievable with folded flexible intraocular lenses.

The cross sectional area of a flexible implant lens or a dehydrated expansile implant lens therefore limits the incision size to a length greater than that required to perform the procedure in modern cataract surgery. The surgeon therefore has to widen the incision prior to insertion of the implant lens. This results in greater astigmatism and an increased likelihood of wound leakage than would otherwise be the case. It is therefore desirable to design an intraocular lens implant which can be inserted through incision sizes of 2.5 mm or less.

### SUMMARY OF THE INVENTION

In accordance with a first aspect of the present invention there is provided a method for insertion of an intraocular lens implant in an eye, which comprises obtaining a dehydrated intraocular lens implant in folded condition, inserting the folded dehydrated intraocular lens implant into the eye through an incision in the eye, and allowing the inserted intraocular lens implant to unfold and hydrate in the eye.

In accordance with a second aspect of the present invention there is provided an intraocular lens implant comprised of a polymer, wherein the polymer is flexible and elastic when dehydrated so as to allow the intraocular lens implant to be folded and inserted into an incision in the eye, and wherein the polymer is expansile when hydrated, such that after insertion into the eye, the intraocular lens implant hydrates and expands.

### DETAILED DESCRIPTION OF THE DRAWINGS

The present invention will now be described, by way of example only, with reference to the accompanying drawings, in which:

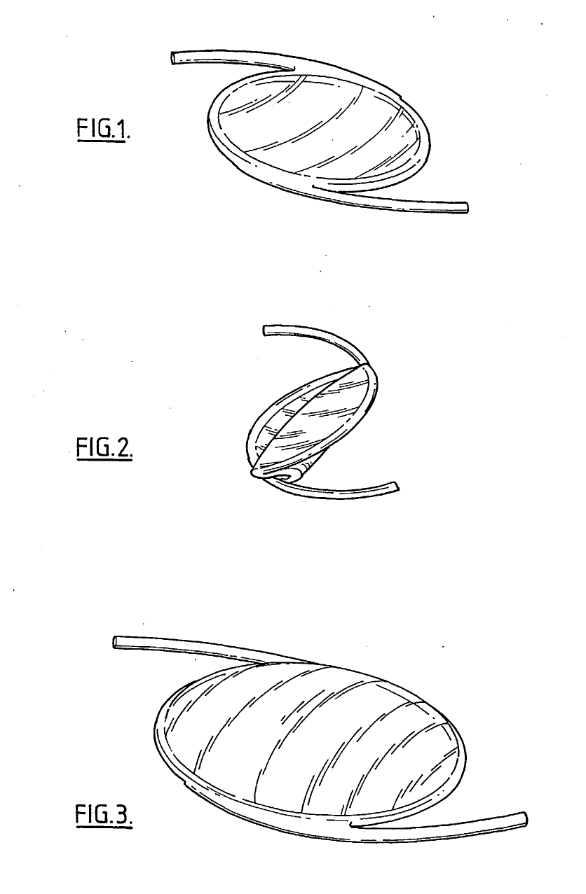

FIG. 1 is a schematic diagram of a dehydrated intraocular lens implant in accordance with the present invention.

FIG. 2 is a schematic diagram of the dehydrated intraocular lens implant of FIG. 1 in a folded configuration before insertion into an incision in an eye; and

FIG. 3 is a schematic diagram of an hydrated expanded intraocular lens implant of FIGS. 1 and 2.

### DETAILED DESCRIPTION OF THE INVENTION

The present invention relates to an intraocular implant lens which may be inserted through a smaller incision than current flexible and expansile implant lenses. In the present invention the lens implant is manufactured from an expansile material which has reduced dimensions when dehydrated (see FIG. 1) and is folded and inserted in the dehydrated state (see FIG. 2). When inserted the lens implant unfolds, hydrates and swells to its final dimensions as shown in FIG. 3. The lens implant therefore utilizes the reduced cross sectional area obtainable by folding as well as the expansile properties to enable insertion through an incision smaller than that otherwise achievable with conventional flexible or expansile lenses.

This result can be achieved by a number of methods of which two are described below by way of example only.

(A) An intraocular lens implant can be manufactured from known hydrogel materials which are rigid when dehydrated and swell and become flexible when hydrated. Polyhydroxyethylmethylmethacryclate (HEMA) is a typical hydrogel material with these properties. A lens implant manufactured from this material can be folded when hydrated and allowed to dry in the folded state. The lens implant can then be supplied in the dehydrated and folded state and implanted through a smaller incision than would otherwise be feasible. A lens implant with a 5.5 mm optic and 1.1 mm centre thickness manufactured from this material will have a reduced diameter of 4.6 mm and thickness of 0.8 mm. When folded such a lens implant can be readily inserted through an incision with a length of 2.5 mm or less.

(B) One disadvantage of a dehydrated rigid expansile lens implant is the time taken to hydrate and unfold. This can be avoided by manufacturing a lens implant from an expansile material which is flexible when dehydrated. An example of a suitable material would be a water insoluble hydrophilic gel comprising a copolymer of water soluble monoolefinic monomers, with or without water insoluble monoolefenic monomers, cross linked with a terminal polyoefinic siloxane macromer. Polysiloxane hydrogels of this nature have been described using 2-hydroxyethylmethacrylate, N-vinyl-2pyrollidone, methylmethacrylate and polydimethylsiloxane as copolymers. A copolymer of glyceryl methacrylate and a siloxane monomer is another example of a suitable polymer that would be flexible when dehydrated yet still have a sufficient water content and swell ratio when hydrated to provide significant expansile properties. A suitable material with a water content of 25 to 35% would have a swell ratio in the order of 1.2. When dehydrated, however, the material would still be flexible.

An intraocular lens implant manufactured from this material could be folded in the dehydrated state prior to insertion at the time of surgery. An implant with an optic diameter of 4.6 mm and thickness of 0.8 mm when dehydrated could be folded and inserted readily through an incision size of less than 2.5 mm. The required incision size would be less than the incision required for a lens implant manufactured from a rigid dehydrated hydrogel material as the material would be compressible during insertion and could also be inserted with the aid of an injector mechanism. Once inserted the lens implant would unfold by nature of the inherent elastic recoil of the polymer then slowly hydrate and acquire the hydrated dimensions of a 5.5 mm optic and centre thickness of 1.1 mm for a 21.5 diopter implant.

Other suitable materials with expansile flexible properties include silicone acrylates, urethane siloxane-acrylates, fumerate end capped siloxanes, and siloxane-hydrogel block prepolymers. Fluorinated siloxane-containing polymers, prepared from fluorinated siloxane-containing monomer and at least one vinyl-or acryl-containing hydrophilic monomer are also suitable materials.

An intraocular lens manufactured from a polymer which is flexible and elastic when dehydrated but expands when hydrated, will be able to be inserted through a smaller incision than a high refractive index foldable lens which is non expansile or an expansile lens which is not flexible. The linear and radial expansion ratio of a hydrogel material varies with the water content. The higher the water content of a hydrogel material the greater the expansion ratio and the lower the refractive index. There will be an upper limit where no further benefit with regard to a reduction in incision size is obtained from increasing the water content and expansion ratio of a material due to the lowering of the refractive index.

The association of expansion ratio, water content and refractive index expected for an expansile flexible material can be estimated by examining the properties of known hydrogel materials. A series of hydrogel polymers of hydroxyethylmethacrylate (HEMA) with increasing glycerol methacrylate as a co-polymer produces materials with the followng water content, expansion ratio and refractive index is provided in Table 1.

TABLE 1

| | Water content | Expansion | Refractive |
|---|---|---|---|
| Material | (Isotonic saline) | ratio | Index |
| PolyHEMA | 38% | 1.2 | 1.44 |
| HEMA | 47% | 1.3 | 1.422 |
| + | | | |
| Glycerol | | | |
| Methacrylate | | | |
| HEMA | 58% | 1.410 | 1.404 |
| + | | | |
| Glycerol | | | |
| Methacrylate | | | |
| HEMA | 68% | 1.55 | 1.383 |
| + | | | |
| Glycerol | | | |
| Methacrylate | | | |

The benefit that can be obtained by reducing the incision size required for an intraocular lens manufactured from an expansile material, which is flexible enough in the dehydrated state so that it can be folded or rolled up and injected through a cylinder, was calculated as follows.

Consider an intraocular lens with a biconvex optic with a power “P”, a diameter “D”, an edge thickness “T” and a refractive index (N). In aqueous the Radius of curvature “R” required for an intraocular lens can be calculated;

R=(N−1.336)*1000/(P/2) Equation 1

The sagittal height of one segment of the biconvex optic “H” can be calculated from the radius of curvature;

H=R−SQR(R*R−(D/2)) Equation 2

The cross sectional area “K” of one segment of the optic is calculated from the sagittal height and radius of curvature;

K=(R*R)*(ACOS(R−H)/R)−SQR(2*RH−H*M) Equation 3

The total cross sectional area of the optic “A” is then;

A=2*K+T*D

Assuming the optic can be folded or rolled in a circular fashion to allow the smallest profile for insertion. The radius of the circular cross sectional area required for insertion of the implant “C” can be calculated;

C=SQR(K*7/22) Equation 4

The length of incision required to insert the lens “L” is determined by considering the circumference of the circular cross sectional area;

L=22*C/7 Equation 5

The incision length required for an intraocular lens was calculated according to the above mentioned Equations for different swell ratios and refractive index and the calculations are tabulated in Table 2. The calculations demonstrated that an intraocular lens manufactured from an expansile flexible material would be less than that required for non-expansile flexible lenses even those with a high refractive index. Similarly the incision size required for an intraocular lens manufactured from an expansile flexible material is less than that required for an expansile lens which is non flexible and can not be folded. Due to the inverse relationship of refractive index and swell ratio the optimum water content appeared to be in the range of 35% to 65% with a range of swell ratios from 1.2 to 1.5.

The optic and haptic of the intraocular lens implant may be manufactured from the same material as a single piece unit or the haptic may be attached to the optic by a variety of mechanisms. Alternatively, only the optic may be manufactured from a material which is flexible and has expansile properties whilst the haptic may be manufactured from conventional materials such as polymethylmethacrylate or polypropylene. There may be one or a plurality of haptics attached to the optic, although the most common configuration includes an optic with two outwardly extending haptics. The purpose of the haptic is to provide optimal centration of the optic as well as a means of fixation of the lens implant within a capsular bag remnant of the original lens following cataract or lens extraction.

It is also possible to implant a folded, expansile lens in front of the capsular bag behind the iris with the haptics resting in the region between the root of the iris and ciliary processes, known as the ciliary sulcus. Intraocular lenses of this type may also be inserted in phakic eyes to correct refractive errors in front of the crystalline lens behind the iris with the haptic providing support in the cilairy sulcus. Furthermore, as an alternative site of implantation in phakic eyes, folded, expansile intraocular lenses may be inserted in front of the iris in the anterior chamber with the haptics resting in the angle of the anterior chamber. The haptic can be supported/fixated on the iris.

Modifications and variations such as would be apparent to a skilled addressee are deemed within the scope of the present invention.

TABLE 2

| Power | | Edge | | | Refrac- | |
|---|---|---|---|---|---|---|
| of | Optic | Thick- | Water | Swell | tive | Length of |
| Lens | Diameter | ness | Content | Ratio | Index | Incision |
| 21 | 6 | 0.2 | 0 | 1 | 1.55 | 2.977971654 |
| 21 | 6 | 0.2 | 0 | 1 | 1.47 | 4.069096439 |
| 21 | 6 | 0.2 | 38% | 1.2 | 1.44 | 2.81139621 |
| 21 | 6 | 0.2 | 47% | 1.3 | 1.42 | 2.593421115 |
| 21 | 6 | 0.2 | 58% | 1.41 | 1.404 | 2.4047006352 |
| 21 | 6 | 0.2 | 68% | 1.55 | 1.383 | 2.579167599 |
| 21 | 5.5 | 0.2 | 0 | 1 | 1.55 | 2.468047615 |
| 21 | 5.5 | 0.2 | 0 | 1 | 1.47 | 3.303851718 |
| 21 | 5.5 | 0.2 | 38% | 1.2 | 1.44 | 2.28052593 |
| 21 | 5.5 | 0.2 | 47% | 1.3 | 1.42 | 2.090101465 |
| 21 | 5.5 | 0.2 | 58% | 1.41 | 1.404 | 1.922617004 |
| 21 | 5.5 | 0.2 | 68% | 1.55 | 1.383 | 2.004591988 |
| 21 | 5 | 0.2 | 0 | 1 | 1.55 | 2.026847916 |
| 21 | 5 | 0.2 | 0 | 1 | 1.47 | 2.651652686 |
| 21 | 5 | 0.2 | 38% | 1.2 | 1.44 | 1.82853391 |
| 21 | 5 | 0.2 | 47% | 1.3 | 1.42 | 1.665363928 |
| 21 | 5 | 0.2 | 58% | 1.41 | 1.404 | 1.519963179 |
| 21 | 5 | 0.2 | 68% | 1.55 | 1.383 | 1.544567108 |
| 28 | 6 | 0.2 | 0 | 1 | 1.55 | 3.582931232 |
| 28 | 6 | 0.2 | 0 | 1 | 1.47 | 5.078277705 |
| 28 | 6 | 0.2 | 38% | 1.2 | 1.44 | 3.536886687 |
| 28 | 6 | 0.2 | 47% | 1.3 | 1.42 | 3.326362665 |
| 28 | 6 | 0.2 | 58% | 1.41 | 1.404 | 3.180451524 |
| 28 | 6 | 0.2 | 68% | 1.55 | 1.383 | 4.043923077 |
| 28 | 5.5 | 0.2 | 0 | 1 | 1.55 | 2.931983944 |
| 28 | 5.5 | 0.2 | 0 | 1 | 1.47 | 4.072175598 |
| 28 | 5.5 | 0.2 | 38% | 1.2 | 1.44 | 2.827197152 |
| 28 | 5.5 | 0.2 | 47% | 1.3 | 1.42 | 2.63516955 |
| 28 | 5.5 | 0.2 | 58% | 1.41 | 1.404 | 2.484841373 |
| 28 | 5.5 | 0.2 | 68% | 1.55 | 1.383 | 2.90257864 |
| 28 | 5 | 0.2 | 0 | 1 | 1.55 | 2.374024963 |
| 28 | 5 | 0.2 | 0 | 1 | 1.47 | 3.222868907 |
| 28 | 5 | 0.2 | 38% | 1.2 | 1.44 | 2.231922451 |
| 28 | 5 | 0.2 | 47% | 1.3 | 1.42 | 2.062372251 |
| 28 | 5 | 0.2 | 58% | 1.41 | 1.404 | 1.920768152 |
| 28 | 5 | 0.2 | 68% | 1.55 | 1.383 | 2.116857852 |

1. A method for insertion of an intraocular lens implant in an eye, which comprises obtaining a dehydrated intraocular lens implant in folded condition, inserting the folded dehydrated intraocular lens implant into the eye through an incision in the eye and allowing the inserted intraocular lens implant to unfold and hydrate in the eye.

2. The method accordidng to claim 1, characterised in that the incision in the eye is 2.5 mm or less.

3. The method according to claim 1 or claim 2, characterised in that a diameter and a thickness of the intraocular lens implant, when dehydrated, are less than the diameter and the thickness of the intraocular lens implant when hydrated in the eye.

4. The method according to claim 2 or claim 3, characterised in that a radius of the dehydrated introcular lens implant is less than 2.5 mm.

5. The method according to claim 2 or claim 3, characterised in that a net thickness of the folded dehydrated intraocular lens implant is less than 2.5 mm.

6. An intraocular lens implant comprised of a polymer, wherein the polymer is flexible and elastic when dehydrated so as to allow the intraocular lens implant to be folded and inserted into an incision in the eye, and wherein the polymer is expansile when hydrated, such that after insertion into the eye, the intraocular lens implant hydrates and expands.

7. The intraocular lens implant according to claim 6, characterised in that an optic of the intraocular lens implant is comprised of the polymer.

8. The intraocular lens implant according to claim 6 or claim 7, characterised in that the polymer comprises hydrogel materials.

9. The intraocular lens implant according to any one of claims 6 to 8, characterised in that a water content of the polymer is about 25 to 65% w/w, and swell ratio of the polyer is about 1.2 to 1.5.

10. The intraocular lens implant according to any one of claims 6 to 9, characterised in that the polymer is a water insoluble hydrophilic gel comprising a copolymer of water soluble monoolefinic monomers, with or without water insoluble monoolefinic monomers, cross linking with a terminal polyolefinic siloxane macromer.

11. The intraocular lens implant according to claim 10, characterised in that the copolymer is selected from a group including 2 hydroxyethylmethacrylate, N-vinyl-2pyrollidone, methylmethacrylate and polydimethylsiloxane.

12. The intraocular lens implant according to claim 10, characterised in that the copolymer is glyceryl methacrylate and a siloxane monomer.

13. The intracular lens implant according to any one of claims 6 to 9, characterised in that the polymer is selected from a group including silicone acrylates, uerthane-siloxane-acrylates, fumerate endcapped siloxanes, and siloxane-hydrogel block prepolymers.

14. The intraocular lens implant according to any one of claims 6 to 9, characterised in that the polymer is a fluorinated siloxane-containing monomer and at least one vinyl-or acryl-containing hydrophilic monomers.
